(19) United States
(12) Patent Application Publication
(10) Pub. No.: US 2024/0309460 A1
(43) Pub. Date: Sep. 19, 2024

(54) USE OF REAGENT FOR DETECTING PIR-HSA-120522 IN PLASMA IN PREPARATION OF LUNG CANCER SCREENING OR DIAGNOSIS KIT

(71) Applicant: WEST CHINA HOSPITAL, SICHUAN UNIVERSITY, Chengdu, Sichuan (CN)

(72) Inventor: Jingsi DONG, Chengdu, Sichuan (CN)

(21) Appl. No.: 18/575,172

(22) PCT Filed: May 10, 2022
(86) PCT No.: PCT/CN2022/091993

§ 371 Date: Dec. 28, 2023

(30) Foreign Application Priority Data
Jun. 29, 2021 (CN) 202110732434.0

(51) Int. Cl. C12Q 1/6886; C12Q 1/686

(52) U.S. Cl. CPC C12Q 1/6886; C12Q 1/686; C12Q 2600/158

(57) ABSTRACT

A reagent for detecting piR-hsa-120522 in plasma is used in the preparation of a non-small cell lung cancer screening or diagnosis kit. Compared with benign nodules and healthy people, piR-hsa-120522 has a significantly higher expression level in plasma of patients with non-small cell lung cancer and precancerous lesions (P<0.05), which indicates that the purpose of screening or diagnosing patients with non-small cell lung cancer can be achieved by means of detecting the expression level of piR-hsa-120522 in plasma.

### FIELD OF THE INVENTION

The present invention belongs to the field of in vitro diagnostic reagents, and specifically relates to the use of reagents for detecting piR-hsa-120522 in plasma in the manufacturer of screening or diagnostic kits for lung cancer and/or its precancerous lesions.

### BACKGROUND OF THE INVENTION

Lung cancer, one of the most common malignant tumors, has the highest mortality rate among all malignant tumors around the world. Lung cancer is also included in the malignant tumors which have the fastest growth in the incidence rate and mortality as well as the greatest threat to human health and life. In the past 50 years, it has been reported that the incidence rate and mortality of lung cancer have increased significantly in many countries, which in men rank first in all malignant tumors, while in women rank second. The pathogenesis of lung cancer has not yet been fully clarified, and many research data suggest that smoking, air pollution, chronic lung inflammation, and long-term exposure to asbestos, dust, metal substances (such as nickel and chromium) and the same may all contribute to the occurrence of lung cancer. At the same time, lung cancer is also related to genetic factors, the body's autoimmune status, and the metabolism.

From the perspective of clinical characteristics, currently, lung cancer is classified internationally as small cell lung cancer (SCLC) and non-small cell lung cancer (NSCLC). NSCLC accounts for more than 85% of lung cancer, while more than 85% of non-small cell lung cancer is found to be advanced lung cancer. Therefore, the diagnosis of lung cancer, especially non-small cell lung cancer, is of great clinical significance.

The examination of non-small cell lung cancer in clinical practice is performed by X-ray, bronchoscopy, screening of carcinoembryonic antigen (CEA) indicator, lavage/sputum cytology, and low-dose spiral CT (LDCT), which has been widely used in recent years. But these screening or diagnostic methods often have many problems, including low specificity and sensitivity, invasive screening or diagnosis, certain radiation damage, and low diagnostic compliance. Therefore, it is still urgently needed to develop an efficient, accurate and non-invasive way to screen or diagnose high-risk groups of non-small cell lung cancer in large-scale, help reduce the incidence rate of non-small cell lung cancer, and improve the early screening rate of non-small cell lung cancer.

In recent years, liquid biopsy techniques based on molecular diagnosis have received widespread attention in tumor diagnosis. The detection of some tumor specific molecular markers such as carcinoembryonic antigen (CEA), squamous cell carcinoma antigen (SCC-Ag), and circulating microRNA (microRNA), combined with auxiliary imaging examinations, can improve the sensitivity of diagnosis. These molecular markers can be directly detected in blood and other body fluids, and thus has certain advantages, such as simplicity, speed, non-invasive, affordability, and reproducibility. These methods are easily accepted by patients and have important clinical significance for tumor diagnosis, thereby possessing good clinical application prospects. Although it has been reported that some molecular markers can be used for the diagnosis of lung cancer, overall, the specificity and reliability of these markers are poor. Therefore, it is further urgent to investigate new efficient and specific molecular markers of lung cancer, that may be used for early diagnosis of non-small cell lung cancer.

At present, there have been no reports of screening or diagnosing lung cancer by detecting the levels of piR-hsa-120522 in plasma.

### SUMMARY OF THE INVENTION

One object of present invention is to provide the use of a reagent for detecting piR-hsa-120522 in plasma in the manufacturer of screening or diagnostic kits for lung cancer and/or its precancerous lesions.

The present invention provides the use of the reagent for detecting piR-hsa-120522 in plasma in the manufacturer of screening or diagnostic kits for lung cancer and/or its precancerous lesions.

Further, the lung cancer is a non-small cell lung cancer.

Further, the reagents are those for PCR, nucleic acid thermal amplification, nucleic acid blotting, colloidal gold assay, nucleic acid sequencing or genechip detection.

Further, the PCR reagent is that for fluorescent quantitative PCR.

Further, the PCR reagent comprises a primer for PCR amplification of piR-hsa-120522, with the primer sequence as represented by the sequences of SEQ ID NO.5 and SEQ ID NO.6.

Further, the PCR reagent further comprises a primer for reverse transcription of piR-hsa-120522, with the primer sequence as represented by the sequence of SEQ ID NO.4.

The present invention provides a screening or diagnostic kit for lung cancer and/or its precancerous lesions, which comprises a reagent for detecting piR-hsa-120522 in plasma.

Further, the reagents are those for PCR, nucleic acid thermal amplification, nucleic acid blotting, colloidal gold assay, nucleic acid sequencing or genechip detection; preferably, the PCR reagent is that for fluorescent quantitative PCR.

Further, the PCR reagent comprises a primer for PCR amplification of piR-hsa-120522, with the primer sequence as represented by the sequences of SEQ ID NO.5 and SEQ ID NO.6.

Further, the PCR reagent further comprises a primer for reverse transcription of piR-hsa-120522, with the primer sequence as represented by the sequence of SEQ ID NO.4.

piR-hsa-120522 consists f a sequence of SEQ ID NO.1, that is GGCGGCCCGGGTTCGACTCCCGGTGTGGGAAC.

The present invention also provides a method for screening lung cancer and/or its precancerous lesions, which comprises the following steps:
(1) Using the above kits to detect piR-hsa-120522 in the plasma of a subject;
(2) Comparison of piR-hsa-120522 in plasma: If the expression level of piR-hsa-120522 in the plasma of a subject is significantly higher than that of the individuals with benign nodules and/or those healthy, the subject can be diagnosed as a patient with lung cancer and/or its precancerous lesions.

Further, the lung cancer is a non-small cell lung cancer.

In the present invention, “significantly higher than” means that the difference is statistically significant.

In the present invention, it has been first discovered that compared with individuals with benign nodules and those healthy, the expression level of piR-hsa-120522 in the plasma of patients with non-small cell lung cancer and precancerous lesions is significantly higher (P=0.032), indicating that detecting the expression level of piR-hsa-120522 in the plasma can achieve the purpose of screening or diagnosing patients with non-small cell lung cancer and precancerous lesions, which is conducive to the early detection of non-small cell lung cancer.

In the present invention, it has been first discovered that compared with the population with benign nodules, the expression level of piR-hsa-120522 in the plasma of patients with non-small cell lung cancer and precancerous lesions is significantly higher (P=0.017), indicating that detecting the expression level of piR-hsa-120522 in the plasma can achieve the purpose of screening or diagnosing patients with non-small cell lung cancer and precancerous lesions, and can effectively distinguish between benign nodules and malignant tumor nodules.

In the present invention, it has been first discovered that compared with individuals with benign nodules and those healthy, the expression level of piR-hsa-120522 in the plasma of patients with non-small cell lung cancer is significantly higher (P=0.048), indicating that detecting the expression level of piR-hsa-120522 in plasma can achieve the purpose of screening or diagnosing patients with non-small cell lung cancer, which is of great significance for effective screening or diagnosis of non-small cell lung cancer.

The screening or diagnostic kit provided in the present invention can quickly, efficiently, and accurately screen or diagnose patients with lung cancer (especially non-small cell lung cancer), providing a new choice for screening or diagnostic kits of lung cancer (especially non-small cell lung cancer).

Obviously, based on the above content of the present invention, according to the common technical knowledge and the conventional means in the field, other various modifications, alternations, or changes can further be made, without department from the above basic technical spirits.

With reference to the following specific examples, the above content of the present invention is further illustrated. But it should not be construed that the scope of the above subject matter of the present invention is limited to the following examples. The techniques realized based on the above content of the present invention are all within the scope of the present invention.

### DESCRIPTION OF FIGURES

FIG. 1. ROC curves for detecting the expression levels of piR-hsa-120522 in the plasma of patients with non-small cell lung cancer and precancerous lesions, as well as individuals with benign nodules and those healthy.

FIG. 2. ROC curves for detecting the expression levels of piR-hsa-120522 in the plasma of patients with non-small cell lung cancer and precancerous lesions, as well as individuals with benign nodules.

FIG. 3. ROC curves for detecting the expression levels of piR-hsa-120522 in the plasma of patients with non-small cell lung cancer, as well as individuals with benign nodules and those healthy.

### EXAMPLES

The raw materials and equipment used in the present invention were known products obtained by purchasing those commercially available.

### Example 1: Composition of Screening or Diagnostic Kits for Non-Small Cell Lung Cancer

The screening or diagnostic kit for non-small cell lung cancer provided in the present invention consists of a reagent for fluorescent quantitative PCR, so as to detect piR-hsa-120522 in human plasma. The reagent for fluorescence quantitative PCR comprises the primers for PCR amplification of piR-hsa-120522, with sequences as represented by SEQ ID NO.5 and SEQ ID NO.6; the reagent also includes the primers for reverse transcription of piR-hsa-120522, with a sequence as represented by SEQ ID NO.4.

### Example 2: Expression/Detection of piR-Hsa-120522 in the Plasma of Patients with Non-Small Cell Lung Cancer

### 1. Clinical Data

73 patients with non-small cell lung cancer, 19 patients with precancerous lesions of non-small cell lung cancer, 49 healthy individuals, and 29 individuals with benign pulmonary nodules were selected.

### 2. Instruments and Reagents

Main instruments: analytikjena-qTOWER2.2 fluorescence quantitative PCR instrument (Germany), SCILOGEX D3024R spinner (USA), ordinary PCR instrument analytikjena-Easycycler (Germany); scandrop100 ultra-trace nucleic acid and protein analyzer (Germany); pipette (Bio-rad, USA).

Main reagents and consumable materials: Reverse transcription kit (TUREScript 1st Stand CDNA SYNTHESIS Kit) (Aidlab); 2×SYBR® Green premix (DF, China). 10 μL pipet tips (GCS, US), 200 μL pipet tips (GCS, US), 1 mL pipet tips (GCS, US), 200 μL RNase-free PCR reaction tube (AXGEN); 1.5 mL RNase-free EP tube (GCS), low profile white PCR reaction tube (bio-rad), optical sealing film (bio-rad); (The pipet tips and EP tube were sterilized and dried for later use).

### 3. Detection Method

Plasma samples were collected from patients with non-small cell lung cancer, precancerous lesions of non-small cell lung cancer, healthy individuals, and subjects with benign pulmonary nodules. U6 was used as the internal reference gene to detect the target gene (primer information is listed in Table 1). SYBR GREEN I was used to detect the expression level of the target gene in 168 plasma samples submitted for assay. The specific steps were as follows:
(1) Extraction of total RNA from samples
(2) RNA assay results

In this experiment, the OD value of RNA was detected using an ultra-trace nucleic acid and protein analyzer (scandrop100), which was the A260/A280 ratio.
(3) Synthesis of the first strand of cDNA

The TUREScript 1st Stand cDNA Synthesis Kit (Aidlab) was used in the procedures of reverse transcription, that was performed in 20 μL of reaction system (different reaction systems were chosen depending on the number of detected genes; in general, the more genes, the larger the reaction system):

reverse transcription of target gene (using a 10 μL reaction system):

| | | |
|---|---|---|
| total RNA | 200 | ng |
| 5 × RT Reaction Mix | 2 | μL |
| RT primer | 0.5 | μL |
| TUREscript H⁻RTase/RI Mix | 0.5 | μL |

| | |
|---|---|
| RNase Free dH₂O | to 10 μL |

The reaction conditions for reverse transcription were as follows:

| | |
|---|---|
| 25° C. | 10 min |
| 42° C. | 50 min |
| 65° C. | 15 min |

After completion of the reaction, cDNA was obtained and stored at −80° C.

Reverse transcription of internal reference gene (using a 10 μL system):

| | | |
|---|---|---|
| total RNA | 200 | ng |
| 5 × RT Reaction Mix | 2 | μL |
| Olig(dT) | 0.5 | μL |
| TUREscript H⁻RTase/RI Mix | 0.5 | μL |

| | |
|---|---|
| RNase Free dH₂O | to 10 μL |

The reaction conditions for reverse transcription were as follows:

| | |
|---|---|
| 42° C. | 45 min |
| 65° C. | 15 min |

After completion of the reaction, cDNA was obtained and stored at −80° C.
(4) Information on primer synthesis

TABLE 1
Primer information.

| Sequence | | |
|---|---|---|
| No. | Primers | Sequence (5′ to 3′) |
| SEQ | U6-F | CTCGCTTCGGCAGCACATATACT |
| ID | | |
| NO. 2 | | |
| SEQ | U6-R | ACGCTTCACGAATTTGCGTGTC |
| ID | | |
| NO. 3 | | |
| SEQ | piR-hsa- | GTCGTATCCAGTGCAGGGTCCGA |
| ID | 120522- | GGTATTCGCACTGGATACGACGT |
| NO. 4 | RT | TCCC |
| SEQ | piR-hsa- | TCGACTCCCGGTGTGGG |
| ID | 120522- | |
| NO. 5 | F1 | |
| SEQ | General | CAGTGCAGGGTCCGAGGTAT |
| ID | downstream | |
| NO. 6 | primers | |

(5) Real time quantitative PCR (QPCR) reaction

The QPCR reaction solution was prepared with the components shown in Table 2, and centrifuged at 4° C. and 6000 rpm for 30 seconds in a PCR plate spinner. Then, the solution was placed in a quantitative PCR instrument, and amplification was performed according to the following procedures.

Fluorescence quantitative PCR program:
step 1. 95° C.-3 min,
step 2. 95° C.-10 min,
step 3. 60° C.-30 s+reading the plate,
return to step 2 and perform 39 cycles,
melting curve analysis (60° C.-95° C., +1° C./cycle, for 4 s).

TABLE 2
Components of QPCR reaction solution.

| Components | Final concentration | Amount (μL) |
|---|---|---|
| 2 × SYBR ®Green Supermix | 1× | 5 |
| Forward primer | 200 nM | 0.5 |
| Reverse primer | 200 nM | 0.5 |
| cDNA | N/A | 1 |
| ddH₂O | N/A | 3 |

(6) Calculation of relative expression level of target genes

The formula for calculating the relative expression level of target genes in each sample was as

$$Ratio = \frac{{( 1 + {E}_{target} )}^{Δ {Ct}_{target ( control - expt )}}}{{( 1 + {E}_{reference} )}^{Δ {Ct}_{reference ( control - expt )}}}$$

(7) Data Processing

Processing was performed using SPSS version 20.0 software. The data were represented by mean±standard deviation, t-test was used for comparison between two groups, and one-way ANOVA was used for comparison among multiple groups. A difference of P<0.05 was statistically significant.

### 4. Experimental Results

Using U6 as the internal reference, the expression levels of target genes in plasma samples of each group were calculated using the 2\hat{ }-ΔΔCT method. The results are shown in Table 3.

TABLE 3
Assay results for expression levels of piR-hsa-
120522 in plasma of different individuals.

| | Average value | Number of | Standard deviation |
|---|---|---|---|
| Groups | (copies/μL) | individuals | (copies/μL) |
| Benign nodules | 433356.4828 | 29 | 387102.83723 |
| Non-small cell lung | 3641103.7397 | 73 | 12897098.21908 |
| cancer | | | |
| Healthy individuals | 895167.0612 | 49 | 982456.80468 |
| Precancerous lesions | 2275360.2632 | 19 | 2890112.94336 |
| Total | 2050726.4199 | 170 | 8344341.44948 |

Further analysis of the expression levels of target genes in plasma samples between patients with non-small cell lung cancer and precancerous lesions, individuals with benign nodules, and those healthy is shown in Table 4.

TABLE 4
Assay results for expression levels of piR-hsa-120522 in the plasma
of patients with non-small cell lung cancer and precancerous
lesions, individuals with benign nodules, and those healthy.

| | Average value | Number of | Standard | Mean standard | P |
|---|---|---|---|---|---|
| Groups | (copies/μL) | individuals | deviation | error | value |
| Non-small | 3359048.0217 | 92 | 11557120.71025 | 1204913.12654 | 0.032 |
| cell lung | | | | | |
| cancer and | | | | | |
| precancerous | | | | | |
| lesion | | | | | |
| Benign | 723468.2564 | 78 | 840622.14735 | 95181.71518 | |
| nodules and | | | | | |
| healthy | | | | | |
| individuals | | | | | |

As shown in Table 4, compared with individuals with benign nodules and those healthy, the expression level of piR-hsa-120522 in the plasma of patients with non-small cell lung cancer and precancerous lesions was significantly higher (P=0.032), indicating that detecting the expression level of piR-hsa-120522 in plasma could achieve the purpose of screening or diagnosing patients with non-small cell lung cancer and precancerous lesions, which was conducive to the early detection of non-small cell lung cancer.

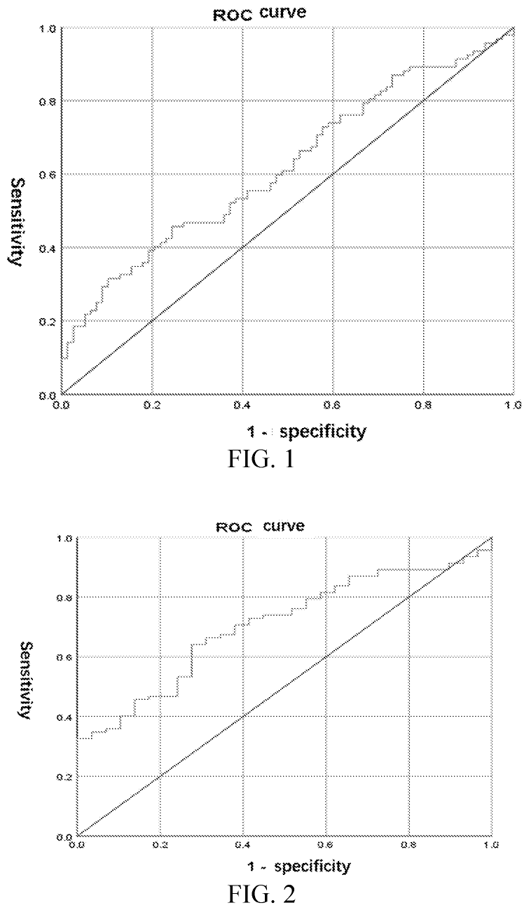

According to the ROC curve (FIG. 1), the area under the ROC curve is 0.619, indicating that the method of screening or diagnosing non-small cell lung cancer and precancerous lesions by detecting the expression level of piR-hsa-120522 in plasma had excellent accuracy, sensitivity, and specificity.

Further analysis of the expression levels of target genes in plasma samples between patients with non-small cell lung cancer and precancerous lesions and individuals with benign nodules is shown in Table 5.

TABLE 5
Assay results for expression levels of piR-hsa-120522 in the
plasma of patients with non-small cell lung cancer and precancerous
lesions and individuals with benign nodules.

| | Average value | Number of | Standard | Mean standard | P |
|---|---|---|---|---|---|
| Groups | (copies/μL) | individuals | deviation | error | value |
| Non-small | 3359048.0217 | 92 | 11557120.71025 | 1204913.12654 | 0.017 |
| cell lung | | | | | |
| cancer and | | | | | |
| precancerous | | | | | |
| lesion | | | | | |
| Individuals | 433356.4828 | 29 | 387102.83723 | 71883.19227 | |
| with benign | | | | | |
| nodules | | | | | |

As shown in Table 5, compared with the individuals with benign nodules, the expression level of piR-hsa-120522 in the plasma of patients with non-small cell lung cancer and precancerous lesions was significantly higher (P=0.017), indicating that detecting the expression level of piR-hsa-120522 in the plasma could achieve the purpose of screening or diagnosing patients with non-small cell lung cancer and precancerous lesions, and could effectively distinguish between benign nodules and malignant tumor nodules.

According to the ROC curve (FIG. 2), the area under the ROC curve is 0.705, indicating that the method of screening or diagnosing patients with non-small cell lung cancer and precancerous lesions by detecting the expression level of piR-hsa-120522 in plasma had excellent accuracy, sensitivity, and specificity.

Further analysis of the expression levels of target genes in plasma samples of patients with non-small cell lung cancer, individuals with benign nodules, and those healthy is shown in Table 6.

TABLE 6
Assay results for expression levels of piR-hsa-120522 in the plasma of patients with
non-small cell lung cancer, individuals with benign nodules, and those healthy.

| | Average value | Number of | Standard | Mean standard | P |
|---|---|---|---|---|---|
| Groups | (copies/μL) | individuals | deviation | error | value |
| Non-small cell | 3641103.7397 | 73 | 12897098.21908 | 1509491.17106 | 0.048 |
| lung cancer | | | | | |
| Benign | 723468.2564 | 78 | 840622.14735 | 95181.71518 | |
| nodules and | | | | | |
| healthy | | | | | |
| individuals | | | | | |

According to Table 6, compared with individuals with benign nodules and those healthy, the expression level of piR-hsa-120522 in the plasma of patients with non-small cell lung cancer was significantly higher (P=0.048), indicating that detecting the expression level of piR-hsa-120522 in plasma could achieve the purpose of screening or diagnosing patients with non-small cell lung cancer, which was of great significance for diagnosis of non-small cell lung cancer.

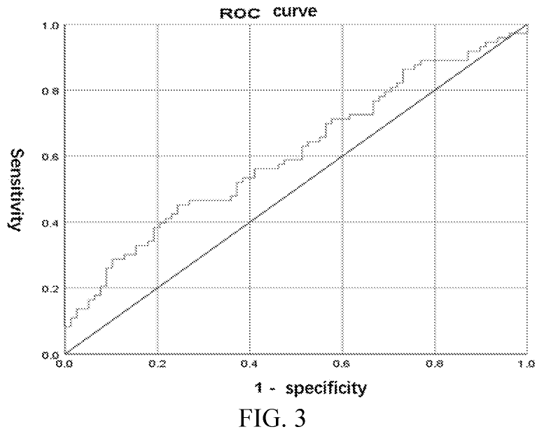

According to the ROC curve (FIG. 3), the area under the ROC curve is 0.608, indicating that the method of screening or diagnosing patients with non-small cell lung cancer by detecting the expression level of piR-hsa-120522 in plasma had excellent accuracy, sensitivity, and specificity.

In conclusion, the present invention provided the use of a reagent for detecting piR-hsa-120522 in plasma in the manufacturer of a screening or diagnosis kit for non-small cell lung cancer. In the present invention, it has been first discovered that compared with individuals with benign nodules and those healthy, the expression level of piR-hsa-120522 in the plasma of patients with non-small cell lung cancer and precancerous lesions was significantly higher (P<0.05), indicating that detecting the expression level of piR-hsa-120522 in the plasma could achieve the purpose of screening or diagnosing patients with non-small cell lung cancer and precancerous lesions, which was of great significance for the effective screening or diagnosis of non-small cell lung cancer. The reagent for detecting piR-hsa-120522 in plasma could be used in the manufacturer of screening or diagnostic kits for non-small cell lung cancer, achieving effective screening or diagnosis of non-small cell lung cancer. The screening or diagnostic kit provided in the present invention could quickly, efficiently, and accurately screen or diagnose patients with lung cancer (especially non-small cell lung cancer), providing a new choice for screening or diagnostic kits for lung cancer (especially non-small cell lung cancer).

1. The use of the reagent for detecting piR-hsa-120522 in plasma in the manufacturer of screening or diagnostic kits for lung cancer and/or its precancerous lesions.

2. The use according to claim 1, characterized in that the lung cancer is a non-small cell lung cancer.

3. The use according to claim 1, characterized in that the reagents are those for PCR, nucleic acid thermal amplification, nucleic acid blotting, colloidal gold assay, nucleic acid sequencing or genechip detection.

4. The use according to claim 3, characterized in that the PCR reagent is that for fluorescent quantitative PCR.

5. The use according to claim 3, characterized in that the PCR reagent comprises a primer for PCR amplification of piR-hsa-120522, with the primer sequence as represented by the sequences of SEQ ID NO.5 and SEQ ID NO.6.

6. The use according to claim 5, characterized in that the PCR reagent further comprises a primer for reverse transcription of piR-hsa-120522, with the primer sequence as represented by the sequence of SEQ ID NO.4.

7. A screening or diagnostic kit for lung cancer and/or its precancerous lesions, characterized in that it comprises a reagent for detecting piR-hsa-120522 in plasma.

8. The screening or diagnostic kit according to claim 7, characterized in that the reagents are those for PCR, nucleic acid thermal amplification, nucleic acid blotting, colloidal gold assay, nucleic acid sequencing or genechip detection; preferably, the PCR reagent is that for fluorescent quantitative PCR.

9. The screening or diagnostic kit according to claim 8, characterized in that the PCR reagent comprises a primer for PCR amplification of piR-hsa-120522, with the primer sequence as represented by the sequences of SEQ ID NO.5 and SEQ ID NO.6.

10. The screening or diagnostic kit according to claim 9, characterized in that the PCR reagent further comprises a primer for reverse transcription of piR-hsa-120522, with the primer sequence as represented by the sequence of SEQ ID NO.4.

11. A method for screening lung cancer and/or its precancerous lesions, characterized in that the method comprises the following steps:
(1) Using the kit according claim 7 to detect piR-hsa-120522 in the plasma of a subject;
(2) Comparison of piR-hsa-120522 in plasma: If the expression level of piR-hsa-120522 in the plasma of a subject is significantly higher than that of the individuals with benign nodules and/or healthy individuals, the subject can be diagnosed as a patient with lung cancer and/or its precancerous lesions.

12. The method according to claim 11, characterized in that the lung cancer is a non-small cell lung cancer.


SEQUENCE LISTING (6 sequences)

SEQ ID NO: 1 - DNA, 32 bp, Artificial sequence
  synthesized
ggcggcccgggttcgactcccggtgtgggaac

SEQ ID NO: 2 - DNA, 23 bp, Artificial sequence
  synthesized
ctcgcttcggcagcacatatact

SEQ ID NO: 3 - DNA, 22 bp, Artificial sequence
  synthesized
acgcttcacgaatttgcgtgtc

SEQ ID NO: 4 - DNA, 50 bp, Artificial sequence
  synthesized
gtcgtatccagtgcagggtccgaggtattcgcactggatacgacgttccc

SEQ ID NO: 5 - DNA, 17 bp, Artificial sequence
  synthesized
tcgactcccggtgtggg

SEQ ID NO: 6 - DNA, 20 bp, Artificial sequence
  synthesized
cagtgcagggtccgaggtat